(19) United States
(12) Patent Application Publication
(10) Pub. No.: US 2017/0119640 A1
(43) Pub. Date: May 4, 2017

(54) Methods of Improving Glucose Tolerance, Hair Regrowth, and Bio-Availability

(71) Applicants: Shil Kothari, Fairfield, CA (US); Gary Troxel, Fairfield, CA (US)

(72) Inventors: Shil Kothari, Fairfield, CA (US); Gary Troxel, Fairfield, CA (US)

(21) Appl. No.: 14/924,721

(22) Filed: Oct. 28, 2015

(51) Int. Cl. A61K 8/43; A61Q 7/00

(52) U.S. Cl. CPC A61K 8/43; A61Q 7/00

(57) ABSTRACT

Methods of improving glucose tolerance in a mammal, improving hair growth and re-growth in a mammal and improving the bio-availability of resveratrol in a mammal by providing a mammal in need of 10 mg to 500 mg per kilogram of body weight of mammal of a resveratrol-arginine conjugate.

### BACKGROUND OF THE INVENTION

Field of the Disclosure

The present disclosure generally relates to methods for increasing glucose tolerance, hair regrowth and providing a more bio-available resveratrol. Specifically, the present disclosure provides methods and related compositions involving a conjugate of resveratrol and amino acid arginine to provide, among other things, increased glucose tolerance, hair regrowth and providing a more bio-available resveratrol.

### GENERAL BACKGROUND

Aging is a natural process with many various physical, physiological and metabolic changes. These changes are accompanied by a range of age-related diseases. Current concerns have focused on the treatment and management of these diseases, not as much on their prevention. There exists a need for increased focus on the prevention of these diseases.

Possible prevention pathways could be linked to our lifestyle and diets. Resveratrol is one possible prevention pathway. Resveratrol is a polyphenolic compound. Certain foods contain resveratrol, such as peanuts, grapes, red wine and some berries. However, the high cost and lack of bio-availability has limited its use as a functional food because it is rapidly metabolized and eliminated. Thus, there also exists a need for an increase in the bio-availability of resveratrol.

Certain foods contain glucose, which is a carbohydrate or a monosaccharide and also has effects on related diseases. Glucose tolerance levels decrease with age and certain diets. Such intolerance could result in higher than normal blood glucose levels or hyperglycemia. This decrease in tolerance levels increases the possibly of other related diseases, such as diabetes. There exists yet another need to increase glucose tolerance levels in mammals.

As a person ages or through certain diets or lifestyles, hair loss can occur. There are many products on the market that claim to regrow hair. There exists a need to significantly improve the rate of hair growth.

### SUMMARY

In one embodiment, a resveratrol-arginine conjugate is provided to provide hair growth and re-growth is provided using a resveratrol-arginine conjugate.

In another embodiment of the present disclosure, a method for improving glucose tolerance is provided using resveratrol and arginine and a resveratrol-arginine conjugate.

In yet another embodiment of the present disclosure, a method for improving hair regrowth is provided using resveratrol and arginine and a resveratrol-arginine conjugate.

In yet another embodiment of the present disclosure, a method for improving bio-availability of resveratrol is provided using resveratrol and arginine and a resveratrol-arginine conjugate.

### DETAILED DESCRIPTION

To determine the efficacy of resveratrol-arginine conjugate, six month old female Albino Wistar rats were divided into seven groups (six rats per group) and given the following daily treatment for four months:

### Group 1: Control;

Group 2: 25 mg ResArgin/kg body wt;
Group 3: 50 mg ResArgin/kg body wt;
Group 4: 75 mg ResArgin/kg body wt;
Group 5: 25 mg Resveratrol/kg body wt;
Group 6: 50 mg Resveratrol/kg body wt;

Group 7: 75 mg Resveratrol/kg body wt. In one embodiment, glucose tolerance levels were tested via a glucose tolerance test and analysis was performed. Compared to the Control group, glucose tolerance was improved in the ResArgin and resveratrol groups (p<0.01 and p<0.05 respectively) over the experimental period.

In one embodiment, glucose tolerance tests were performed after having the rats fast for 6 hours and injecting 2 g/kg body weight intraperitoneally. Blood was then collected from the tail vein at 0, 15, 30, 60 and 120 minutes after injection.

In another embodiment, hair regrowth assay was done and quantified. Compared to the Control group, hair regrowth was improved in the ResArgin and resveratrol groups over the experimental period.

In the hair regrowth assay, dorsal hair was removed by plucking from a square of approximately 1.5 cm×1.5 cm. Hair growth and re-growth was scored two weeks later on the basis of digital photographs.

In yet another embodiment, the present disclosure resveratrol is made more bio-available because of the conjugation of resveratrol and the amino acid arginine.

ResArgin and resveratrol significantly improved the investigated age related conditions, with the effects of ResArgin being more significant due to its composition or its improved bio-availability resulting from the presence of arginine. Based on the results of the study performed, the effective dosage of resveratrol arginine conjugate is 10 to 250 mg per kilogram of body weight for a period of at least two weeks.

1. A method of growing hair in a mammal, the method comprising:
providing to a mammal in need thereof 10 mg to 500 mg per kilogram of body weight of the mammal of resveratrol and arginine in tablet, capsule or caplet form for a period of at least two weeks;

2. The method according to claim 1, wherein the resveratrol and arginine is a resveratrol-arginine conjugate.

3-5. (canceled)

6. The method according to claim 1, wherein the period of at least two weeks is at least four weeks.

7. A method comprising:
providing a mammal in need of hair growth or re-growth 10 mg to 500 mg per kilogram of body weight of the mammal of resveratrol and arginine in tablet, capsule or caplet form for a period of at least two weeks.

8. The method according to claim 7, wherein the resveratrol and arginine is a resveratrol-arginine conjugate.

9-11. (canceled)

12. The method according to claim 7, wherein the period of at least two weeks is at least four weeks.

13. A method of hair re-growth comprising:
administering to a mammal 10 mg to 500 mg per kilogram of body weight of the mammal of resveratrol and arginine in tablet, capsule or caplet form for a period of at least two weeks.

14. The method according to claim 13, wherein the resveratrol and arginine is a resveratrol-arginine conjugate.

15-17. (canceled)

18. The method according to claim 13, wherein the period of at least two weeks is at least four weeks.

19. The method according to claim 13, wherein the period of at least two weeks is at least six weeks.
